(19) United States
(12) Patent Application Publication
(10) Pub. No.: US 2026/0300831 A1
(43) Pub. Date: Oct. 1, 2026

(54) SYSTEMS AND METHODS FOR ITERATIVE MACHINE LEARNING MODEL OPTIMIZATION THROUGH ADAPTIVE PARAMETER ADJUSTMENT

(71) Applicant: AML JV, LLC, Aurora, CO (US)

(72) Inventors: Md Nazmul ISLAM, Aurora, CO (US); Clayton SMITH, Aurora, CO (US)

(73) Assignee: AML JV, LLC, Aurora, CO (US)

(21) Appl. No.: 19/631,708

(22) Filed: Mar. 27, 2026

Related U.S. Application Data
(60) Provisional application No. 63/780,997, filed on Mar. 31, 2025.

(51) Int. Cl. G06N 20/00; G16H 10/60

(52) U.S. Cl. CPC G06N 20/00; G16H 10/60

(57) ABSTRACT

Described herein are systems and methods that related to techniques for iterative machine learning model optimization through adaptive parameter adjustment. In an example, a system can be configured to obtain input data that includes at least one user input specifying a type of endpoint. Based on the input data, the system can identify features from a set of features to use in evaluating multiple models, with each feature corresponding to diagnostic criteria used to represent aspects of treatment profiles maintained in a database. The system can train and evaluate performance of each of the models and generate a user interface to display performance metrics for the models. In some examples, this user interface can be provided to a device that generated the input data to show the performance metrics for the models.

### CROSS-REFERENCE TO RELATED APPLICATIONS

This application claims priority to U.S. Provisional Patent Application Ser. No. 63/780,997, filed Mar. 31, 2025, which is incorporated by reference in its entirety for all purposes.

### TECHNICAL FIELD

This application generally relates to techniques for iterative machine learning model optimization through adaptive parameter adjustment and, in some embodiments, to techniques for iterative machine learning model optimization through adaptive parameter adjustment when modeling responses based on administration of an agent.

### BACKGROUND

Currently, diseases such as acute myeloid leukemia (AML) are treated in coordination with treatment plans developed by clinicians and based on standard of care therapies. These treatment plans can include the use of therapies such as administration of agents targeting specific molecules or pathways involved in the growth and survival of the disease, stem cell transplants, and/or the like. Typically, these treatments are administered in conjunction with monitoring of the response of the patient through diagnostic testing and updated to optimize a patient's outcome. But it is often difficult for clinicians to determine which therapy will be most effective in treating the targeted disease. This can lead to therapies being applied to patients that are less efficient and that do not result in an optimal outcome.

### SUMMARY

Various risk stratification approaches can be implemented for newly diagnosed patients, such as elderly AML patients. However, these approaches may not directly account for real-world data problems and assume that risk models behave similarly across all real-world data environments. This can result in essentially a one-size-fits-all approach, which may not be able to account for variability in a patient's clinical data. Because these approaches result in models that are static in nature, the ability of the models to be tuned is limited and often dependent on periodic (e.g., yearly) updates. Additionally, it can be difficult to gauge and assess the performance of multiple, competing risk models (including similar models with varying configurations). This can lead to such systems implementing oversimplified or outdated risk models that do not reflect current patient needs.

To address the real-world data challenges presented, and improve efficiency in risk model development, techniques have been established that allow for the development of a series of dynamic and context-specific machine learning-based risk models to subdivide AML patients into subgroups (e.g., adverse, intermediate, favorable) based on their expected response to a specific therapy involving one or more agents (e.g., venetoclax (VEN) and azacitidine (AZA)). In some implementations, these systems can allow for the creation of risk models with real-time tuning of context-specific parameters using a retrospective healthcare dataset, such as a dataset established by global patient databases and refined datasets as described in detail below. These systems can be configured to receive input from users at remote computing devices (e.g., using point-and-click tools established by a user interface) that, by virtue of their configuration, reduce or eliminate the need for hardcoding coding certain parameters when testing model configurations. As a result, systems described can also allow for users to more quickly and dynamically develop risk models as compared to other systems. In some examples, the systems described can allow for the generation and comparison of the newly-developed model configurations with competing models (e.g., ELN22, ELN24, Refined ELN24, mPRS, e-mPRS, and MRS) to allow for effective comparison of these models based on multiple statistical parameters, such as equitability, separability, conformity, and predictability. Further, patient data can be updated to at least partially de-identify the patient(s) involved, allowing for the patient data to be processed by remote systems to classify new patients into risk groups without sharing of personally identifiable health information.

The techniques described herein address the technical challenges associated with conventional risk stratification approaches by providing a dynamic, parameter-driven framework for iterative model optimization. Conventional risk stratification systems often rely on static models that are updated infrequently and do not account for variability in real-world clinical data environments. Such systems typically employ fixed feature sets and predefined endpoints, resulting in models that cannot be readily adapted to new clinical scenarios or evolving treatment protocols without extensive recoding or recompilation. The techniques described herein can overcome such limitations by enabling real-time configuration of endpoint-specific criteria, feature selection, and model parameters through user-defined inputs. The analytics server can dynamically compute endpoint-specific risk differences for candidate features at user-specified time-points, selecting only those features that satisfy minimum risk-difference thresholds. The analytics server can parameterize models without recompiling underlying code, such that the models can be iteratively trained and evaluated based on adaptive diagnostic criteria. The analytics server can execute multiple models concurrently on treatment profiles filtered according to endpoint criteria and selected features, generating performance metrics that quantify separability, conformity, and predictability across risk groups. The analytics server can generate user interfaces that present comparative visualizations of model performance, including heatmaps encoding performance metric values over multiple time-points, enabling users to assess model effectiveness under varying clinical conditions. By implementing such techniques, the analytics server can reduce computational overhead associated with manual model reconfiguration, minimize network communications required for dataset transfers, and improve accuracy of risk predictions by tailoring models to specific clinical endpoints and patient populations. The techniques described herein can provide a stateless training architecture that automatically reevaluates models as new treatment profiles become available, such that model performance remains current without requiring persistent state management or hardcoded parameter updates. The analytics server can process de-identified treatment profiles obtained from remote databases, enabling collaborative model development across multiple research organizations while preserving patient privacy. The techniques described herein can thereby provide a technical improvement over conventional approaches by enabling rapid, iterative optimization of machine learning models through adaptive parameter adjustment, resulting in more precise and context-specific risk stratification for clinical decision-making.

In examples, a system is described that is configured to obtain input data associated with at least one user input indicating an endpoint type (indicating survival, event-free survival, etc., at 5, 10, 15 months, etc.) and determine one or more criteria corresponding to an endpoint based on the endpoint type. The system can determine one or more features from among a plurality of features to use when evaluating a plurality of models. Each feature can correspond to diagnostic criteria included in a database representing a plurality of treatment profiles. In some examples, the system can configure a first model, and a second model based on the one or more criteria and the one or more features to generate a first output and a second output. The first output can be generated by the first model and the second output can be generated by the second model. In response to executing the first model and the second model, the system can be configured to generate a user interface comprising an indication of a first performance metric value for the first model and a second performance metric for the second model. The first performance metric can represent the output of the first model and the second performance metric can represent the output of the second model. The system can then provide the user interface to a device involved in generating the input data to indicate the first performance metric value and the second performance metric value.

By implementing the techniques described herein systems can be configured for the rapid manipulation of machine learning model configurations, allowing for quick testing and comparison of performance in a model development environment (e.g., prior to fully training the models). For example, iterative model testing can allow reduced processor or memory consumption by focusing computational resources on preliminary evaluations rather than detailed model training, thereby optimizing resource allocation during the development phase. In at least some examples, iterative testing can also reduce network communications by minimizing the need to transfer large datasets or fully trained models across distributed systems, as only partial or preliminary data exchanges are necessary. Additionally, the techniques described can allow for the selection and generation of more precise outputs by tailoring models to specific tasks or domains, improving accuracy compared to generalized models that are less specialized for targeted predictions.

In one embodiment, a system for iterative machine learning model optimization through adaptive parameter adjustment can include one or more processors. The system can obtain input data associated with at least one user input indicating an endpoint type. The system can determine one or more criteria corresponding to an endpoint based on the endpoint type. The system can determine one or more features from among a plurality of features to use when evaluating a plurality of models, each feature corresponding to diagnostic criteria included in a database representing a plurality of treatment profiles. The system can configure a first model and a second model based on the one or more criteria and the one or more features to generate a first output and a second output, the first output generated by the first model and the second output generated by the second model. In response to executing the first model and the second model, the system can generate a user interface including an indication of a first performance metric value for the first model and a second performance metric for the second model, the first performance metric representing the output of the first model and the second performance metric representing the output of the second model. The system can provide the user interface to a device involved in generating the input data to indicate the first performance metric value and the second performance metric value.

In some implementations, the system can determine a period of time associated with the endpoint type. In some implementations, in response to execution of one or more operations to update the first model and the second model to cause the first model and the second model to generate outputs in accordance with the period of time, the system can obtain the first model and the second model from a device involved in updating the first model and the second model.

In some implementations, the plurality of treatment profiles can include a first plurality of treatment profiles. In some implementations, the system can update the first model and the second model using a training database representing a second plurality of treatment profiles.

In some implementations, the system can obtain a plurality of treatment profiles representing states of patients being treated. In some implementations, the system can execute one or more operations to update one or more aspects each treatment profile of the plurality of treatment profiles to de-identify at least a portion of each treatment profile. In some implementations, the system can generate the training database in response to de-identifying the at least a portion of each treatment profile.

In some implementations, the system can determine a plurality of features, each feature of the plurality of features corresponding to an element of the diagnostic criteria. In some implementations, the diagnostic criteria can correspond to at least one of chromosomal analysis, flow cytometry, fluorescence in situ hybridization, or next generation sequencing.

In some implementations, the system can determine one or more features to be excluded that are represented by the plurality of treatment profiles. In some implementations, the system can determine the plurality of features based on the input and the excluded features.

In some implementations, the system can obtain the first model and the second model from among a plurality of models, the plurality of models including a first risk stratification model configured to output overall survival rates, or a second risk stratification model configured to output event-free survival rates.

In some implementations, the system can obtain patient data associated with a supplemental patient database from a device involved in generating the input data associated with at least one user input. In some implementations, the system can update the plurality of treatment profiles based on the supplemental patient database to supplemental treatment profiles included in the supplemental patient database.

In some implementations, the system can generate a first heatmap based on the first performance metric value and a second heatmap based on the second performance metric. In some implementations, the system can generate the user interface based on the first heatmap and the second heatmap.

In another embodiment, a method for iterative machine learning model optimization through adaptive parameter adjustment can be performed, for example, by one or more processors coupled to non-transitory memory. The method can include obtaining input data associated with at least one user input indicating an endpoint type. The method can include determining one or more criteria corresponding to an endpoint based on the endpoint type. The method can include determining one or more features from among a plurality of features to use when evaluating a plurality of models. Each feature can correspond to diagnostic criteria included in a database representing a plurality of treatment profiles. The method can include executing a first model and a second model based on the one or more criteria and the one or more features to generate a first output and a second output, the first output generated by the first model and the second output generated by the second model. In response to executing the first model and the second model, the method can include generating a user interface including an indication of a first performance metric value for the first model and a second performance metric for the second model, the first performance metric representing the output of the first model and the second performance metric representing the output of the second model. The method can include providing the user interface to a device involved in generating the input data to indicate the first performance metric value and the second performance metric value.

In some implementations, determining the one or more criteria corresponding to the endpoint can include determining a period of time associated with the endpoint type. In some implementations, the method can further include, in response to execution of one or more operations to update the first model and the second model to cause the first model and the second model to generate outputs in accordance with the period of time, obtaining the first model and the second model from a device involved in updating the first model and the second model.

In some implementations, the plurality of treatment profiles can include a first plurality of treatment profiles. In some implementations, the method can further include updating the first model and the second model using a training database representing a second plurality of treatment profiles.

In some implementations, updating the first model and the second model using the training database can include obtaining a plurality of treatment profiles representing states of patients being treated. In some implementations, updating can include executing one or more operations to update one or more aspects each treatment profile of the plurality of treatment profiles to de-identify at least a portion of each treatment profile. In some implementations, the method can include generating the training database in response to de-identifying the at least a portion of each treatment profile.

In some implementations, determining the one or more features can include determining a plurality of features. In some implementations, each feature of the plurality of features can correspond to an element of the diagnostic criteria, where the diagnostic criteria corresponds to at least one of chromosomal analysis, flow cytometry, fluorescence in situ hybridization, or next generation sequencing.

In some implementations, determining the plurality of features can include determining one or more features to be excluded that are represented by the plurality of treatment profiles. In some implementations, determining can include determining the plurality of features based on the input and the excluded features.

In some implementations, executing the first model and the second model can include obtaining the first model and the second model from among a plurality of models, the plurality of models including a first risk stratification model configured to output overall survival rates, or a second risk stratification model configured to output event-free survival rates.

In some implementations, the method can further include obtaining patient data associated with a supplemental patient database from a device involved in generating the input data associated with at least one user input. In some implementations, the method can include updating the plurality of treatment profiles based on the supplemental patient database to supplemental treatment profiles included in the supplemental patient database.

In some implementations, generating the user interface can include generating a first heatmap based on the first performance metric value and a second heatmap based on the second performance metric. In some implementations, generating can include generating the user interface based on the first heatmap and the second heatmap.

In yet another embodiment, one or more non-transitory computer-readable mediums storing instructions, when executed by one or more processors, can cause the one or more processors to obtain input data associated with at least one user input indicating an endpoint type. The instructions can cause the one or more processors to determine one or more criteria corresponding to an endpoint based on the endpoint type. The instructions can cause the one or more processors to determine one or more features from among a plurality of features to use when evaluating a plurality of models, each feature corresponding to diagnostic criteria included in a database representing a plurality of treatment profiles. The instructions can cause the one or more processors to configure a first model and a second model based on the one or more criteria and the one or more features to generate a first output and a second output, the first output generated by the first model and the second output generated by the second model. In response to executing the first model and the second model, the instructions can cause the one or more processors to generate a user interface including an indication of a first performance metric value for the first model and a second performance metric for the second model, the first performance metric representing the output of the first model and the second performance metric representing the output of the second model. The instructions can cause the one or more processors to provide the user interface to a device involved in generating the input data to indicate the first performance metric value and the second performance metric value.

In some implementations, the plurality of treatment profiles can include a first plurality of treatment profiles. In some implementations, the instructions can further cause the one or more processors to update the first model and the second model using a training database representing a second plurality of treatment profiles.

It is to be understood that both the foregoing general description and the following detailed description are exemplary and explanatory and are intended to provide further explanation of the embodiments described herein.

### BRIEF DESCRIPTION OF THE DRAWINGS

The accompanying drawings constitute a part of this specification, illustrate one or more embodiments and, together with the specification, explain the subject matter of the disclosure.

FIG. 1 is a block diagram of an environment, in accordance with one or more embodiments described herein.

FIG. 2 is a flow diagram illustrating operations of a method for modeling responses based on administration of an agent, in accordance with one or more embodiments described herein.

FIG. 3 is a flow diagram illustrating operations of a method for iterative machine learning model optimization through adaptive parameter adjustment, in accordance with one or more embodiments described herein.

FIG. 4 is an example of a system architecture for implementing iterative machine learning model optimization through adaptive parameter adjustment, in accordance with one or more embodiments described herein.

FIG. 5 is an example of a user interface provided to generate user input used when implementing iterative machine learning model optimization through adaptive parameter adjustment, in accordance with one or more embodiments described herein.

FIG. 6 is an example of a heatmap generated to indicate model performance over time, in accordance with one or more embodiments described herein.

### DETAILED DESCRIPTION

Reference will now be made to the embodiments illustrated in the drawings, and specific language will be used here to describe the same. It will nevertheless be understood that no limitation of the scope of the disclosure is thereby intended. Alterations and further modifications of the features illustrated here, and additional applications of the principles as illustrated here, which would occur to a person skilled in the relevant art and having possession of this disclosure, are to be considered within the scope of the disclosure.

This disclosure relates to techniques for addressing the mismatch between training data from a synthetic domain and training data of a real-world domain to improve model performance on real-world datasets and applications. Machine learning models can be used in a variety of different applications, including but not limited to autonomous vehicle applications, robots, or industrial applications. To operate in different settings, machine learning models are trained using a diverse set of domain-specific data, such that the machine learning models can accurately process data from real-world environments having those different settings. Due to the scarcity of available training data for certain real-world scenarios, various systems often rely on synthetically generated data (“synthetic data”) for training machine learning models. For example, for object detection applications, synthetic data may include computer-generated images with corresponding labels that identify objects and their locations in the corresponding images.

However, because synthetic data is generated using algorithmic approaches, there is often a mismatch between the distribution represented by synthetic data and the actual distribution of real-world data for a given application. Conventional approaches to addressing the mismatch between synthetic data and real-world data involve modifying loss functions or model architectures, which are highly specific to particular applications or settings. Success in one application does not necessarily transfer to others. Other conventional approaches can involve translating, or modifying, the synthetic data to better conform to the distribution (or domain) of real-world data. However, such approaches to translate synthetic data to the real-world domain do not perform corresponding modifications to ground-truth labels, resulting in a mismatch between the modified synthetic data and its corresponding ground-truth label. For example, if a synthetic image is modified to be more realistic, the labels applicable to the synthetic version of the image will often require adjustments as well, such as changes to the shape or dimensions of an object.

The techniques described herein provide techniques to directly change synthetic labels based on raw images or metadata, such that the modified labels better correspond to the domain of real-world data. To do so, the techniques described herein can employ a generative adversarial network (GAN)-based model to translate labels from the synthetic domain to the real-world domain. The GAN model can include a generator network that can produce new or changed data and a discriminator network that can evaluate the data to distinguish between real or synthetic data. The generator network can be trained to produce synthetic data that the discriminator network cannot distinguish from real data, such that the synthetic labels are sufficiently realistic.

To implement these techniques, a GAN model can be trained on synthetic or real-world datasets. The GAN model can use images or metadata, such as bounding regions or depth information, to translate synthetic labels to match the labeling distribution in real-world datasets. Once trained, the GAN model can be applied to synthetic labels to obtain modified labels that are similar to real-world labels. The modified labels can replace the original synthetic labels to generate an updated training dataset. The techniques described herein modify the training dataset rather than the downstream model that is to be trained using the modified training dataset, and translate labels instead of images, minimizing the joint distribution between source or target datasets.

In doing so, the techniques described herein can generate training datasets that result in improved downstream performance of various machine learning models, including image object detectors, which rely on the size or quality of labeled datasets. By combining reducing synthetic label mismatches, the techniques described herein provide a more consistent and accurate labeling approach that improves the alignment between synthetic and real-word datasets. The techniques described herein can be particularly useful in scenarios where annotating bounding boxes is ambiguous, such as when objects are occluded, truncated, or crowded. The training datasets generated using the techniques described herein improve downstream machine learning models without require application-specific loss function or architecture modifications, providing a technical improvement over existing approaches.

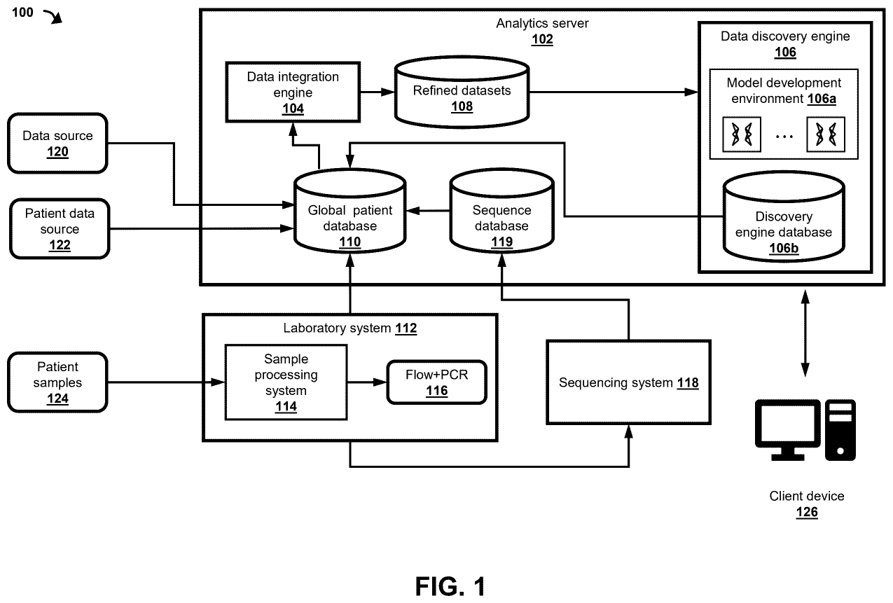

FIG. 1 is a block diagram of an environment 100 for managing patient data, according to an embodiment. The environment 100 can include an analytics server 102, a laboratory system 112, a sequencing system 118, a data source 120, patient data source 122, patient samples 124, and a client device 126. Various components depicted in FIG. 1 can belong to an organization involved in clinical research of one or more diseases such as, for example, acute myeloid leukemia (AML) or other diseases and/or to one or more organizations involved in treating patients with the one or more diseases. While certain components and devices are illustrated as being included in the environment 100 of FIG. 1, it will be understood that the environment 100 is not confined to the components or diseases as described herein and can include additional or different components (not shown for purposes of brevity and clarity) which are configured to be considered within the scope of the embodiments described herein.

In some embodiments, the analytics server 102 can include any computing device comprising a processor and non-transitory machine-readable storage capable of executing the various tasks, processes, and/or operations as described herein. The analytics server 102 can employ various processors such as central processing units (CPUs), graphical processing units (GPUs), and/or the like. Some non-limiting examples of such computing devices can include workstation computers, laptop computers, server computers, and/or the like. While the environment 100 includes a single analytics server 102, there can be multiple analytics servers 102. Further, the analytics server 102 can include any number of computing devices operating in a distributed computing environment such as, for example, a cloud computing environment. As described herein, the analytics server 102 can include a data integration engine 104, a data discovery engine 106, refined datasets 108, a global patient database 110, and a sequence database 119. In some embodiments, the analytics server 102 can include and/or implement operations that are associated with the laboratory system 112, the sequencing system 118, and/or the client device 126. In some embodiments, the analytics server 102 can include and/or implement operations that are associated with (e.g., involved in the generation of) the data source 120, the patient data source 122, and/or the patient samples 124.

In some embodiments, the analytics server 102 can be configured to receive data from the data source 120, the patient data source 122, and the laboratory system 112 and sequencing system 118 when processing patient samples 124. For example, the analytics server 102 can be configured to receive data from the data source 120, where the data is associated with (e.g., represents) entries corresponding to one or more patient files. As an example, as patients interact with clinicians, the clinicians can generate information that are received as input at a client device (not explicitly illustrated) that is associated with the clinicians, the notes indicating clinical observations and/or updates to treatment plans for the patients made by the clinician. The client device can then generate patient data that is associated with each patient and representative of the clinical observations or updates to the treatment plans and store the patient data in the data source 120 to later transmit to the analytics server 102. In this example, the analytics server 102 can implement the global patient database 110 such that the patient data is uploaded and stored in the global patient database 110 in association with one or more identifiers for the patient as described herein.

In another example, the analytics server 102 can be configured to receive data from the patient data source 122, where the data is associated with (e.g., represents) information about individual patients. As an example, as a history of a patient is obtained, the clinicians and/or the patients can generate information that is received as input at a client device (not explicitly illustrated) that is associated with the clinicians and/or patients, the information indicating aspects of the history of the patient such as whether the patient is associated with a history of a given disease in their family, whether the patient had any exposure to environmental conditions associated with the given disease, and/or the like. The client device can then generate patient data that is associated with each patient and representative of the history of the patient and store the patient data in the patient data source 122 to later transmit to the analytics server 102. In this example, the analytics server 102 can obtain and store the patient data in the global patient database 110 in association with one or more identifiers for the patient as described herein.

In yet another example, the analytics server 102 can be configured to receive data from the laboratory system 112 and/or the sequencing system 118, where the data is associated with (e.g., represents) information about patient samples (e.g., tissue samples, blood samples, blood counts (e.g., complete blood counts), bone marrow aspiration and biopsy results, lumbar puncture results, and/or the like) as well as the results of the processing of the samples (e.g., a DNA sequence or targets thereof). As an example, as a patient is evaluated and/or treated for a disease such as AML, patient samples 124 similar to those described above can be obtained. The patient samples 124 can be initially obtained and processed by a laboratory system 112 and processed by a sample processing system 114. The sample processing system 114 can implement one or more devices configured to obtain and store the patient samples and extract DNA from the patient samples. For example, in preparation for genetic analysis to guide AML treatment, patient blood or bone marrow can first be obtained from a patient and frozen. Later, these samples can be quality checked to ensure the sample purity and quantity are sufficient for sequencing. In some embodiments, the isolated DNA can then undergo further processing to be separated into manageable fragments and equipped with adapters (e.g., short, specific pieces of synthetic DNA associated with the fragmented DNA molecules) for compatibility with sequencing machines. In some embodiments, the samples can also be provided to a flow and polymerase chain reaction (PCR) system to extract and amplify the isolated DNA. The laboratory system 112 can then provide the processed samples and corresponding data representing the samples to be processed by the sequencing system 118. Additionally, or alternatively, the laboratory system 112 can then provide the data generated by the laboratory system 112 when processing the samples to the analytics server 102 to be stored in the global patient database 110.

In some embodiments, the sequencing system 118 can be configured to receive the patient samples and/or the isolated DNA and sequence the patient samples. In one example, the sequencing system 118 can attach DNA fragments to a surface in a specific pattern, creating clusters. The sequencing itself can involve a series of cycles where fluorescently labeled nucleotides are introduced one by one. The incorporation of each base can be detected, identifying the sequence of the fragment base by base. Finally, the sequencing system 118 can analyze the vast amount of data, assemble the original DNA sequences, and identify any variations or mutations present (sometimes referred to as Next-Generation Sequencing (NGS)). The sequencing system 118 can then provide data associated with the sequenced DNA to the analytics server 102. In this example, the analytics server 102 can store the sequenced DNA in a sequence database 119 that stores the sequenced DNA in association with one or more patient identifiers established by the analytics server 102. In some embodiments, the analytics server 102 can also cause the sequence database 119 to provide the data associated with the sequenced DNA to the global patient database 110 to be stored in association with other data associated with the patient such as a treatment profile and/or limited treatment profile for the patient as described herein.

In some embodiments, the analytics server 102 can implement a data integration engine 104 to process data stored in the global patient database 110. For example, the analytics server 102 can implement the data integration engine 104 such that the data integration engine 104 is configured to obtain the data associated with the patients that is stored in the global patient database 110 and processes the data to be used by the data discovery engine 106. In one example, as data is obtained by the global patient database 110 for a given patient, the data can be stored in the global patient database 110 in association with one or more identifiers as part of a profile for the patient. The data integration engine 104 can then obtain the data associated with the patient (e.g., the entire profile or portions thereof) from the global patient database 110 and process the data to generate a limited treatment profile. The limited treatment profile can then be stored in the refined datasets database 108 (referred to herein as “refined datasets”) and made available to the data discovery engine 106. In this way, the analytics server 102 can maintain two separate datasets that allow for updates to the limited treatment profiles stored in the refined datasets 108 and subsequent use by the data discovery engine 106 when performing the operations described herein. As will be understood, in this example the data associated with the patient that is stored in the global patient database 110 can be updated over time such that the patient profile is represented as a set of entries associated with a time series. As the global patient database 110 is updated, the data integration engine 104 can obtain updated versions of the data associated with the patient from the global patient database 110, process the data when updating the limited treatment profiles in the refined datasets 108, and store the updates in the refined datasets 108.

In some embodiments, the analytics server 102 can implement the data discovery engine 106 that includes a model development environment 106a and a discovery engine database 106b. For example, the analytics server 102 can implement the data discovery engine 106 such that the data discovery engine 106 is configured to receive data associated with one or more limited treatment profiles that are stored in the refined datasets 108 and process the one or more limited treatment profiles. In this example, the analytics server 102 can process the one or more limited treatment profiles using the model development environment 106a. Processing the limited treatment profiles can include providing the limited treatment profiles to one or more models (e.g., machine learning-based models and/or the like) to determine one or more metrics (or performance metrics) corresponding to the performance of each of the models to indicate which model is most accurate, efficient, and/or the like at generating one or more predictions. These predictions can include indications of treatment options that have a likelihood of optimizing an outcome (e.g., lifespan) for the patients. Processing the limited treatment profiles can additionally, or alternatively, include determining one or more aspects of the limited treatment profiles. For example, where the limited treatment profiles is associated with a predetermined number of possible attributes but the patient samples 124 obtained to be processed were limited and only usable to determine a subset of the possible attributes, the model development environment 106a can process the portions of the refined patient profile that are available in the refined datasets 108 to determine one or more of the remaining attributes. In this example, data associated with the one or more remaining attributes can be stored by the data discovery engine 106 in the discovery engine database 106b. The analytics server 102 can then periodically or in real-time update the global patient database 110 based on the data associated with the limited treatment profiles (e.g., the one or more remaining attributes and/or the like) that are stored in the discovery engine database 106b.

In some embodiments, the client device 126 can include any computing device comprising a processor and non-transitory machine-readable storage capable of executing the various tasks, processes, and/or operations as described herein. The client device 126 can employ various processors such as central processing units (CPUs), graphical processing units (GPUs), and/or the like. Some non-limiting examples of such computing devices can include workstation computers, laptop computers, server computers, and/or the like. While the environment 100 includes a single client device 126, there can be multiple client devices 126. Further, the client device 126 can include any number of computing devices operating in a distributed computing environment such as, for example, a cloud computing environment. In some embodiments, the client device 126 can be associated with one or more software developers and/or one or more clinicians that are interacting with (e.g., configuring operation of) the analytics server 102 as described herein. In some embodiments, the client device 126 can be associated with one or more clinicians and/or one or more organizations involved in treating patients with the one or more diseases such as a hospital and/or the like.

In some embodiments, the analytics server 102 can generate and display an electronic platform (e.g., via the client device 126) when receiving and processing patient data associated with one or more patients, performing one or more operations when analyzing the patient data, and outputting data associated with the results of the operations performed by any of the components of the analytics server 102 such as, for example, the data discovery engine 106. The electronic platform can include graphical user interfaces (GUI) displayed by display devices of one or more client devices 126. An example of the electronic platform generated and hosted by the analytics server 102 can be a web-based application or a website configured to be displayed on different electronic devices, such as mobile devices, tablets, personal computers, and the like.

The above-mentioned components can be configured to interconnect with to each other and establish communication connections therebetween through a network (not explicitly illustrated). Examples of the network can include, but are not limited to, private or public local-area-networks (LAN), wireless LAN (WLAN) networks, metropolitan area networks (MAN), wide-area networks (WAN), and the Internet. The network can include wired and/or wireless communications according to one or more standards and/or via one or more transport mediums. The communication over the network can be performed in accordance with various communication protocols such as Transmission Control Protocol and Internet Protocol (TCP/IP), User Datagram Protocol (UDP), and IEEE communication protocols. In one example, the network can include wireless communications according to Bluetooth specification sets or another standard or proprietary wireless communication protocol. In another example, the network can also include communications over a cellular network, including, e.g., a GSM (Global System for Mobile Communications), CDMA (Code Division Multiple Access), and EDGE (Enhanced Data for Global Evolution) network.

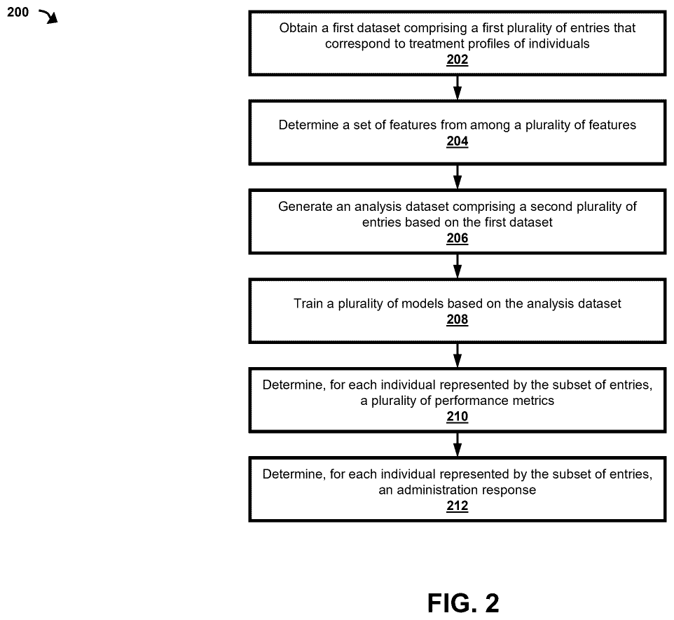

FIG. 2 is a flow diagram illustrating operations of a method 200 for modeling responses based on administration of an agent, in accordance with one or more embodiments described herein. In some implementations, one or more of the functions described with respect to the method 200 can be performed (e.g., completely, partially, and/or the like) by an analytics server that is the same as, or similar to, the analytics server 102 of FIG. 1. In some implementations, one or more of the functions described with respect to the method 200 can be performed (e.g., completely, partially, and/or the like) by another device or group of devices separate from and/or including the analytics server, such as by one or more client devices that are the same as, or similar to, the client device 126 of FIG. 1.

At operation 202, the analytics server can obtain a first dataset comprising a first plurality of entries that correspond to treatment profiles of individuals. For example, the analytics server can obtain a first dataset comprising a first plurality of entries that correspond to treatment profiles of individuals that are diagnosed as having or not having a disease (e.g., AML, etc.). The treatment profiles of the first dataset can include treatment profiles and/or limited treatment profiles as described herein. While the techniques discussed refer to diseases such as AML, it will be understood that they are not limited to AML and that they can be implemented in view of any suitable combination of disease and agent(s).

In some embodiments, each treatment profile can comprise indicators such as biomarkers that are represented as values that quantify a state of an individual represented by the treatment profile. For example, each treatment profile can include indicators captured at a point in time at which an individual is suspected of or diagnosed as having the disease. Additionally, or alternatively, each treatment profile can include information about an age of the individual when the indicators were measured (e.g., through a laboratory system and/or a sequencing system that are the same as, or similar to, the laboratory system and sequencing system of FIG. 1).

In some embodiments, the analytics server can be configured to filter the first plurality of entries based on one or more criteria. For example, the analytics server can filter the first plurality of entries based on criteria indicating administration of at least one therapeutic agent such as venetoclax (VEN) and azacitidine (AZA) to the individuals represented by the first dataset. In this example, the analytics server can be configured to filter the first plurality of entries based on whether the individuals were treated using the at least one therapeutic agent to adjust progression of the disease. In some embodiments, the analytics server can be configured to filter the first plurality of entries based on one or more other criteria. For example, the analytics server can be configured to filter the first plurality of entries based on an age of the individuals represented by the first plurality of entries. In these examples, the analytics server can then determine a second plurality of entries in response to filtering the first plurality of entries.

At operation 204, the analytics server can determine a set of features from among a plurality of candidate features. For example, the analytics server can determine a set of features corresponding to indicators represented by the treatment profiles of the first plurality of entries or the second plurality of entries. In some embodiments, the analytics server can determine the set of features that correspond to indicators generated in accordance with one or more techniques implemented by the laboratory system and/or the sequencing system. In this example, the analytics server can determine the set of features based on measurements and analysis of samples of the individuals obtained and processed by one or more of: flow cytometric systems, cytogenetic systems, fluorescence in situ hybridization systems, next generation sequencing systems, and/or the like.

In some embodiments, the analytics server can compute, for each candidate feature and for an endpoint type associated with a clinical outcome of patients treated with the at least one therapeutic agent, an endpoint-specific risk difference between patients having the feature and patients lacking the feature at a time-point for evaluating the clinical outcome. The analytics server can then select the one or more features as those candidate features whose endpoint-specific risk difference satisfies a minimum risk-difference threshold that specifies a lower boundary for the endpoint-specific risk difference required to designate a candidate feature as clinically significant for the endpoint type. In some implementations, the minimum risk-difference threshold can be expressed as a percentage difference in predicted outcome probabilities between patients exhibiting the candidate feature and patients lacking the candidate feature at the time-point specified by the configuration parameters. For example, where the endpoint type is overall survival and the time-point is twelve months post-treatment, the analytics server 102 can select a candidate feature as one of the one or more features only if the difference in predicted twelve-month survival probability between patients having the candidate feature and patients lacking the candidate feature equals or exceeds the minimum risk-difference threshold (e.g., five percent, ten percent, fifteen percent, etc.). In another example, the analytics server 102 can compute the endpoint-specific risk difference by determining a first survival probability at the time-point for a first subgroup of treatment profiles characterized by presence of the candidate feature, determining a second survival probability at the time-point for a second subgroup of treatment profiles characterized by absence of the candidate feature, and calculating an absolute difference between the first survival probability and the second survival probability, such that the candidate feature satisfies the minimum risk-difference threshold when the absolute difference equals or exceeds a threshold value. The analytics server 102 can apply the minimum risk-difference threshold to filter out candidate features that exhibit minimal prognostic impact, such that the one or more features selected for model parameterization correspond to biomarkers, diagnostic criteria, or clinical indicators demonstrating a quantifiable influence on the clinical outcome at the specified time-point. For example, the analytics server can evaluate each candidate biomarker to determine whether the difference in predicted clinical outcome (e.g., overall survival, event-free survival, etc.) between patients exhibiting the biomarker and those not exhibiting the biomarker exceeds a predefined threshold at the specified time-point. In some embodiments, this selection process can be performed in accordance with one or more configuration parameters obtained from user input, where the configuration parameters include the time-point for evaluating the clinical outcome and the minimum risk-difference threshold.

At operation 206, the analytics server can generate an analysis dataset comprising a second plurality of entries based on the first dataset. For example, the analytics server can generate the analysis dataset based on the first dataset and the set of features identified by the analytics server as satisfying (e.g., being compatible with) a given model to be trained and/or fitted. In one example, where a model is being trained and/or fitted to predict health outcomes (e.g., survival rates at a point or points in time after the individual was treated) based on indicators generated using a particular system and/or technique (e.g., a flow cytometric system), the analytics server can generate a portion of the analysis dataset to use when training and/or fitting that model. In this example, the analytics server can include the features represented in the first plurality of entries that correspond to indicators derived from the flow cytometric systems, cytogenetic systems, fluorescence in situ hybridization systems, next generation sequencing systems, and/or the like.

In some embodiments, the analytics server can augment the analysis dataset to include one or more sub-entries corresponding to each entry. For example, the analytics server can augment the analysis dataset to generate a plurality of sub-entries for one or more of the second plurality of entries, where each sub-entry includes one or more synthetic indicators that augment the indicators for the respective one or more of the second plurality of entries. The analytics server can determine these synthetic indicators based on execution of one or more resampling techniques (e.g., bootstrapping, etc.).

At operation 208, the analytics server can train and/or fit a plurality of models based on the analysis dataset. For example, the analytics server can train a plurality of models based on the entries and/or sub-entries included in the analysis dataset. In this example, the analytics server can determine combinations of entries and/or sub-entries in the analysis dataset that satisfy the configuration of a particular model and train and/or fit that particular model based on the corresponding portions of the analysis dataset. In some embodiments, the analytics server can configure a first model and a second model by parameterizing the first model and the second model to output, for each treatment profile, a risk stratification into a plurality of risk groups for the endpoint type. The analytics server can set model parameters without recompiling model code, based on the endpoint-specific configuration parameters obtained from the client device. This parameterization can enable the analytics server to adapt the models to varying clinical scenarios and evaluation criteria without requiring recompilation or redevelopment of the underlying model architecture.

At operation 210, the analytics server can determine, for each individual represented by the subset of entries, a plurality of performance metrics. For example, the analytics server can determine, for each individual, performance metrics representing predictions of survival at a point in time after disease diagnosis. The analytics server can then determine an average performance metric for each individual. For example, where the analytics server trains and/or fits a plurality of treatment profiles of the analysis dataset to a plurality of models, the analytics server can then determine the metrics for each individual using the plurality of models. The analytics server can then determine a plurality of probabilities that the individual will survive to one or more points in time and average these probabilities to determine an average performance metric.

In some embodiments, the analytics server can determine a treatment response classification for each individual based on the performance metrics associated with that individual. For example, the analytics server can determine an average performance metric corresponding to each individual to determine a treatment response classification. The analytics server can then determine an administration response for each individual based on the average performance metric corresponding to each individual. The treatment response can indicate whether the individual is predicted to have a strongly adverse treatment response, a moderately adverse treatment response, a neutral treatment response (signaling adversity), a neutral treatment response (signaling favorability), a moderately favorable treatment response, or a strongly favorable treatment response.

At operation 212, the analytics server can determine, for each individual represented by the subset of entries, an administration response. For example, the analytics server can determine an administration response for each individual, where the administration response indicates a favorable, intermediate, or not favorable response to treatment with the at least one therapeutic agent.

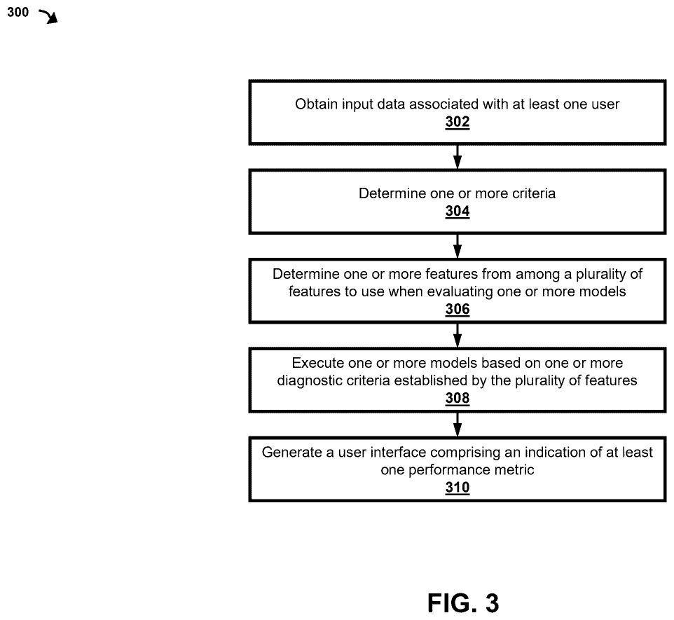

FIG. 3 is a flow diagram illustrating operations of a method 300 for iterative machine learning model optimization through adaptive parameter adjustment, in accordance with one or more embodiments described herein. In some implementations, one or more of the functions described with respect to the method 300 can be performed (e.g., completely, partially, and/or the like) by an analytics server that is the same as, or similar to, the analytics server 102 of FIG. 1. In some implementations, one or more of the functions described with respect to the method 300 can be performed (e.g., completely, partially, and/or the like) by another device or group of devices separate from and/or including the analytics server, such as by one or more client devices that are the same as, or similar to, the client device 126 of FIG. 1.

At operation 302, the analytics server can obtain input data associated with at least one user input. For example, the analytics server can obtain the input data associated with the at least one user input, such as input provided by a clinician, researcher, data scientist, etc., operating a client device (e.g., that is the same as, or similar to, the client device 126 of FIG. 1). The user input can indicate selection of an endpoint type associated with a clinical outcome of patients treated with at least one therapeutic agent. In at least some examples, the user input can also indicate one or more configuration parameters including a time-point for evaluating the clinical outcome and a minimum risk-difference threshold. The analytics server can process the input data to identify and categorize the specified endpoint type and the configuration parameters. As described below, the analytics server can use this endpoint-specific information to iteratively update models to optimize their operation through adaptive parameter adjustment as described herein.

At operation 304, the analytics server can determine one or more criteria. These criteria can be used to control execution of operations by a model development environment (e.g., in a model development environment that is the same as, or similar to, the model development environment 106a of FIG. 1) when training and/or evaluating various models (e.g., statistics-based models, machine learning models, etc.). For example, the analytics server can determine one or more criteria corresponding to an endpoint based on the endpoint type and the one or more configuration parameters indicated in the user input (as represented by the input data). In examples, where the endpoint type is associated with event-free survival, the analytics server can establish criteria such as the absence of disease recurrence, progression, or death at the time-point specified by the configuration parameters. In other examples, where the endpoint type is associated with overall survival, the analytics server can establish similar criteria for survival rates at the specified time-point.

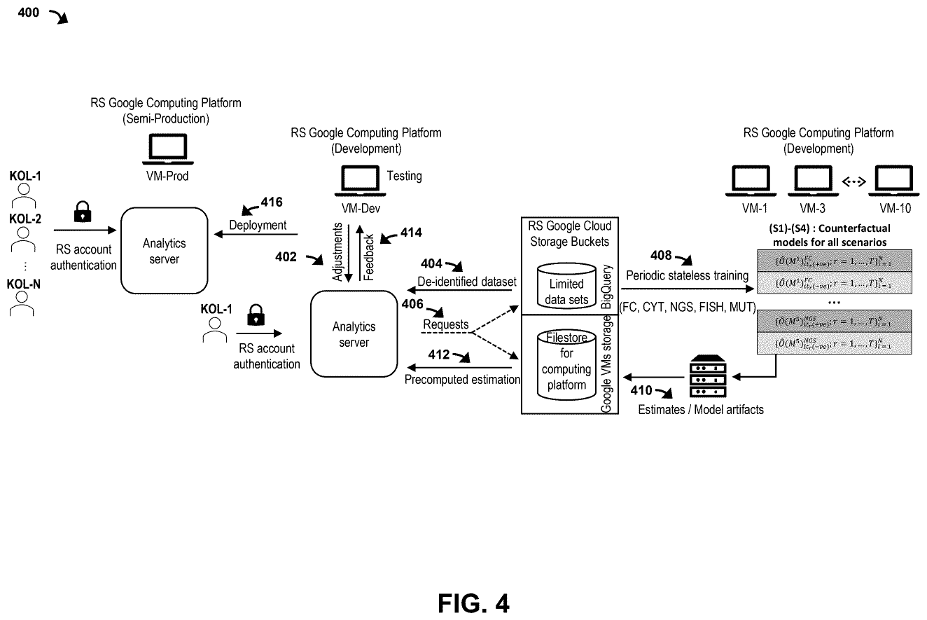

In some embodiments, the analytics server can determine the one or more criteria based on one or more user-configured parameters. These criteria can be used to configure the training and/or evaluation (e.g., performance evaluation) of one or more machine learning models. For example, the analytics server can determine the user-configured parameters based on the user input represented by the input data generated by the client device (e.g., user selections obtained at a client device controlled by the user and involved in the generation of the input data). The user-configured parameters can include specific endpoint configurations (e.g., indicating a time-point of interest at which user wants to maximize the therapeutic outcomes such as overall survival (OS), event-free survival (EFS), etc.), treatment adjustments (e.g., used to filter datasets for model training such as, in the case of AML, whether or not a patient has had one or more stem cell transplants, etc.), risk difference thresholds (e.g., the minimum critical risk difference between adverse features or favorable features), clinical rules (e.g., prioritizing the use of treatment profiles including readily-available genetics or phenotypic features lab results for which testing is more readily available as opposed to specialized lab results (whole genome sequencing, etc.)), approved techniques for addressing missing data (complete case analysis, multiple imputation, inverse probability weighting, likelihood-based analysis, etc.), test type combinations (select combinations of test types for diagnosing and characterizing acute myeloid leukemia (AML), including flow cytometry (FC), next-generation sequencing (NGS), cytogenetic analysis, fluorescence in situ hybridization (FISH), and composite approaches integrating multiple methodologies, composite mutation variable (that combine FISH/NGS/PCR), etc.), and so on. In some embodiments, the analytics server can determine one or more risk profiles (described in detail with respect to the system architecture 400 of FIG. 4). For example, the analytics server can determine the one or more risk profiles based on the one or more criteria described above.

At operation 306, the analytics server can determine one or more features from among a plurality of candidate features to use when evaluating one or more models. For example, the analytics server can determine the one or more features, where each feature corresponds to diagnostic criteria (e.g., values or ranges of values established by biomarkers used to analyze the state of a given patient) represented by the treatment profiles maintained by the global patient database, the refined datasets, and/or a remote database and/or a supplemental patient database (described below). The diagnostic criteria can be represented based on quantifiable outputs derived from chromosomal analysis (e.g., karyotype abnormalities), flow cytometry (e.g., CD7 or CD70 expression levels), fluorescence in situ hybridization (e.g., HER2/CEP17 ratios), or next generation sequencing (e.g., variant allele frequencies), etc.

In some embodiments, the analytics server can determine the one or more features by computing, for each candidate feature and for the endpoint type, an endpoint-specific risk difference between patients having the feature and patients lacking the feature at the time-point specified by the configuration parameters. The analytics server can then select the one or more features as those candidate features whose endpoint-specific risk difference satisfies the minimum risk-difference threshold. For example, the analytics server can evaluate each candidate biomarker to determine whether the difference in predicted clinical outcome (e.g., overall survival, event-free survival, etc.) between patients exhibiting the biomarker and those not exhibiting the biomarker exceeds the minimum risk-difference threshold at the specified time-point. Based on this analysis, the analytics server can select a subset of features (that include, at least in part, biomarker values representing the state of respective patients at one or more points in time) that are clinically relevant and satisfy the user-defined risk-difference criteria.

In another example, the analytics server can determine one or more features to exclude (also referred to as excluded features), where each feature corresponds to diagnostic criteria that, when satisfied by treatment profiles, should result in the exclusion of the treatment profiles during model training and/or evaluation. The exclusionary diagnostic criteria can similarly be represented based on quantifiable outputs derived from chromosomal analysis, flow cytometry, fluorescence in situ hybridization, next generation sequencing, etc. In implementations, the analytics server can compare the user-defined exclusionary criteria received against diagnostic parameters within the treatment profiles to filter out a subset of treatment profiles (that include, at least in part, sets of biomarker values representing the state of respective patients at one or more points in time) that do not meet the desired exclusion parameters. Based on this comparative analysis, the analytics server can exclude a subset of treatment profiles that are not clinically relevant or fail to satisfy the user-defined exclusionary diagnostic criteria.

In some embodiments, the analytics server can determine the features based on determining one or more biomarkers are correlated, or not correlated, with one or more endpoints as described herein. For example, where the analytics server receives input to configure one or more models to classify treatment profiles to indicate whether the patients will satisfy one or more event-free survival, overall survival metrics, etc., the analytics server can execute one or more operations to determine the degree to which various biomarkers and/or therapies are correlated with the respective survival metrics. The analytics server can then determine the biomarkers used to configure the one or more models as described below. A more detailed description of feature selection is described below with respect to operation 404 of FIG. 4.

At operation 308, the analytics server can configure one or more models based on one or more diagnostic criteria established by the plurality of features. For example, the analytics server can configure one or more models when training the one or more models using the treatment profiles that satisfy the diagnostic criteria discussed above. In examples, the analytics server can select the one or more models from among a plurality of models based on a task indicated by the input data, such as classifying treatment profiles in accordance with a risk stratification scheme to indicate that each patient corresponding to each treatment profile will have either a favorable response (e.g., a positive reaction to a specific therapy involving one or more agents, such as venetoclax (VEN) and azacitidine (AZA)), a neutral response (e.g., a reaction to a specific therapy that is neither strongly positive nor strongly negative), or an unfavorable response (e.g., a reaction to a specific therapy that either has no effect or negatively impacts the patient's condition).

In some embodiments, the analytics server can configure a first model and a second model by parameterizing the first model and the second model to output, for each treatment profile, a risk stratification into a plurality of risk groups for the endpoint type. The analytics server can set model parameters without recompiling model code, based on the endpoint-specific configuration parameters obtained from the client device. This parameterization can enable the analytics server to adapt the models to varying clinical scenarios and evaluation criteria without requiring recompilation or redevelopment of the underlying model architecture. Additionally, or alternatively, the analytics server can select the one or more models based on the endpoint types and/or endpoint criteria (e.g., indicating a period of time at which the endpoint is to be set during evaluation, etc.) identified by the input data. For example, the analytics server can select a combination of models selected from risk stratification models that are configured for classification of overall survival rates based on treatment profiles, event-free survival rates, or combinations thereof. In examples, these models can be based on various architectures, including ELN 22 (European LeukemiaNet 2022), ELN 24 (European LeukemiaNet 2024 (Dohner et al. 2024)), Refined ELN 22 (Refined European LeukemiaNet 2024 (Curtis et al 2024)), mPRS (molecular prognostic risk signatures (Dohner et al 2024)), e-mPRS (extended molecular prognostic risk signature (Islam et al 2024)), and/or MRS (risk signatures developed by Mayo-Clinic (Naseema et al. 2024)). In some examples, these models can include semi-parametric regularized models (e.g., hazard models with ridge penalty, etc.). In examples, hazard models compatible with survival data can additionally, or alternatively, be implemented without loss of generality (e.g., deep survival network, random survival forest, or other semi-parametric models with different penalty terms). Additionally, the analytics server can implement deep learning architectures (e.g., artificial neural networks that implement convolutional neural networks, attention-based networks, etc.). For example, the analytics server can execute artificial neural networks that implement feed-forward networks with adjustments for censoring like inverse probability of censoring weights, or a series of multilayer perceptron models ignoring censoring for the processed subsets of the original datasets (which can be observed within a range of time-points).

During implementation (e.g., at inference time), the analytics server can execute the first model and the second model on treatment profiles selected from the database based on the endpoint criteria and the subset of features to generate a first set of model outputs and a second set of model outputs. In some examples, the treatment profiles (including relevant portions of electronic health records (EHRs), laboratory results, genetic profiles, and other biomarkers relevant to the treatment being evaluated) can be preprocessed to remove or update personal health information and/or personally identifiable information before being provided as inputs to the models. In some embodiments, the analytics server can then obtain the outputs of the models, where the output corresponds to the classification of each treatment profile into risk categories such as “favorable,” “neutral,” or “negative” responses.

During training, the analytics server can provide the treatment profiles as inputs to the machine learning models. For supervised models, this can include providing treatment profiles with known outcomes as inputs, comparing the outputs to known outcomes, and iteratively updating and executing the models to cause the outputs to more closely match the known outcomes. This can allow the model(s) to learn the relationship between inputs and outputs once convergence is achieved. During training, the model's parameters (weights) can be iteratively updated using optimization techniques like gradient descent. In instances where the models include machine learning models, backpropagation can also be implemented to calculate the error between predicted and actual outcomes and adjust weights of the models accordingly to minimize this error. The analytics server can also cross-validate the models to confirm that the models generalize to unseen data (e.g., treatment profiles that were not used during training) by splitting the dataset into training and testing subsets. Techniques such as hyperparameter tuning and addressing class imbalance (e.g., through resampling or weighted loss functions) can further be implemented to refine the performance of the models.

In some embodiments, the analytics server can obtain treatment profiles from various sources specified by the input data to use to train and/or evaluate the models. For example, the analytics server can identify one or more databases, including remote databases and/or supplemental patient databases (e.g., maintaining one or more supplemental treatment profiles), specified by user input involved in generating the input data. In at least some examples, the analytics server can establish communication connections with the remote databases using secure protocols and authentication mechanisms, ensuring data privacy and integrity during transmission. The analytics server can then use the treatment profiles obtained from the specified databases to either augment (e.g., add to) the treatment profiles already accessible by the analytics server, or to independently be used to train and evaluate the models maintained by the model development environment. In some embodiments, the remote database can be maintained by different research organizations, medical facilities, etc., monitoring patients being treated for various diseases (e.g., AML and so on).

In some embodiments, the analytics server can obtain or otherwise transform the treatment profiles into limited treatment profiles accessible by the analytics server for training and/or evaluation of the models as described herein. For example, the analytics server can execute operations to de-identify the treatment profiles. This can involve removing user identifiers that uniquely identify the patients represented by each treatment profile, replacing the user identifiers with substitute identifiers that can be mapped to the treatment profiles but do not directly indicate the identity of the patients represented, etc. Additionally, or alternatively, this can involve updating the treatment profiles to adjust parameters of each treatment profile that can be used to identify the patients corresponding to each treatment profile. For example, the analytics server can offset one or more dates consistently across the treatment profile by a period of time (e.g., one month, six months, one year, etc.) to reduce or eliminate the chances of the identity of the patient corresponding to the treatment profile being reidentified based on the dates of the entries in the treatment profile (e.g., the dates associated with one or more diagnostic tests, etc.). In some embodiments, the analytics server can then generate a training database in response to identifying the treatment profiles and use the training database to evaluate one or more models as described herein.

In some embodiments, the analytics server can automatically reevaluate the above-described models periodically by training the base (e.g., untrained) models, and assessing their performance in accordance with the diagnostic criteria described above (causing the analytics server to implement a stateless architecture). For example, as additional treatment profiles are obtained and/or updates to existing treatment profiles are obtained over time, the analytics server can train the base models and evaluate their performance. In an example, the analytics server can execute the base models using the treatment profiles available to the analytics server that satisfy the diagnostic criteria established by the user-defined parameters. During training, the analytics server can select the previously-selected models and/or additional models that are newly-accessible to the analytics server based on the task being performed (e.g., treatment profile classification for risk stratification), train the base models, and evaluate the base models as described above. In some embodiments, this automatic reevaluation can be periodic and can occur weekly, monthly, quarterly, yearly, etc. Once trained, the analytics server can evaluate the performance of each of the models and determine one or more performance metrics represented as one or more performance metric values that can be used to compare model performance. For example, for each of the first model and the second model, the analytics server can compute at least one performance metric value based on the corresponding set of model outputs. The performance metrics can represent the ability of the models to classify treatment profiles in accordance with equitability (e.g., how fairly the model treats different subgroups within the data), separability (e.g., the model's ability to distinguish between different classes or outcomes), conformity (e.g., how well each model's predictions align with actual or known outcomes), and/or predictability (e.g., the accuracy and reliability of the model's predictions). In some embodiments, the performance metric value can include at least one of: a separability metric between the risk groups, a conformity metric between the risk groups and observed clinical outcomes, or a predictability metric at the time-point.

In some implementations, the analytics server 102 can determine a separability metric by computing a statistical measure quantifying the degree to which the risk groups (for example, adverse, intermediate, and/or favorable) occupy distinct regions in a feature space derived from the treatment profiles. For example, the analytics server 102 can calculate a separability metric by evaluating pairwise distances between centroids of the risk groups in a multidimensional space defined by selected biomarkers, such that a higher separability metric indicates greater divergence in the predicted outcomes among the risk groups. In another example, the analytics server 102 can compute the separability metric by determining an area under a receiver operating characteristic (ROC) curve for pairwise comparisons of the risk groups, where a separability metric approaching unity indicates that the first model and/or the second model reliably distinguish between patients assigned to different risk groups based on the endpoint type. The analytics server 102 can determine a conformity metric by quantifying the alignment between risk stratifications output by the first model and/or the second model and observed clinical outcomes recorded in the treatment profiles. For example, the analytics server 102 can compute a conformity metric by comparing observed survival probabilities at the time-point specified by the configuration parameters against model-predicted probabilities for each risk group, such that a conformity metric near zero indicates strong agreement between the model outputs and real-world outcomes. In some implementations, the analytics server 102 can calculate the conformity metric by applying a calibration curve analysis that plots predicted risk probabilities against observed event rates across deciles of the treatment profiles, where deviations from a diagonal line indicate miscalibration and reduce the conformity metric value. The analytics server 102 can determine a predictability metric by evaluating the consistency of the first model and/or the second model in forecasting clinical endpoints across multiple time-points and/or across different subsets of the treatment profiles. For example, the analytics server 102 can compute a predictability metric by measuring a variance in performance metric values (such as concordance indices and/or Brier scores) obtained from cross-validation folds applied to the analysis dataset, such that a lower variance indicates higher predictability and more stable model behavior. In another example, the analytics server 102 can determine the predictability metric by calculating a time-dependent area under the curve (AUC) value at a plurality of time-points (for example, at six months, twelve months, and eighteen months post-treatment), where consistent AUC values across the plurality of time-points indicate that the first model and/or the second model maintains predictive accuracy over the course of disease progression.

At operation 310, the analytics server can generate a user interface comprising an indication of at least one performance metric. For example, in response to executing the first model and the second model, the analytics server can be configured to generate a user interface that includes one or more elements (e.g., charts, graphs, tables, heatmaps, etc.) representing the performance metrics determined for each of the models evaluated by the analytics server. The user interface can comprise an indication of a first performance metric value for the first model and a second performance metric value for the second model, the first performance metric representing the output of the first model and the second performance metric representing the output of the second model. These performance metrics can be represented as global performance metrics (e.g., indicating overall performance of the models) or as time-series performance metrics (e.g., indicating performance over a period of time). In some examples, the user interface can display regression performance metrics such as survival area under the curve (AUC) values, iBrier values, etc.

In some embodiments, the user interface can include interactive elements that allow users to provide input that, when obtained by the analytics server, causes the analytics server to generate updates to the user interface indicating specified aspects of the performance of each of the evaluated models. These specific aspects can include performance across different data subsets or time periods, performance involving certain treatment profiles or treatment profiles satisfying a certain diagnostic criterion (e.g., a subset of the earlier-established diagnostic criteria), etc. The analytics server can update the displayed performance metrics and visualizations in real-time as new evaluation data becomes available, enabling continuous monitoring of model performance.

In some embodiments, the analytics server can generate heatmaps that visually represent the performance metrics indicating model performance over time. For example, the analytics server can collect and aggregate performance metrics indicating various aspects of the performance of each model (e.g., when trained and analyzed using the model development environment). The analytics server can then analyze these data points to generate heatmaps, using color gradients to highlight areas of high and low performance. In some embodiments, the indication of the first performance metric value can be a first heatmap visually encoding the at least one performance metric value of the first model over a plurality of time-points, the indication of the second performance metric value can be a second heatmap visually encoding the at least one performance metric value of the second model over a plurality of time-points, and the user interface can concurrently present the first heatmap and the second heatmap to enable comparative visual evaluation of the first model and the second model. In some embodiments, the analytics server can update these heatmaps over time (e.g., when automatically reevaluating the performance of the models). The analytics server can then generate user interfaces based on these heatmaps that are then provided to the client device causing the evaluation of the models to indicate the results of the evaluation.

In some embodiments, the analytics server can provide the user interface to a device involved in generating the input data to cause the user interface to be displayed by that device. For example, the analytics server can generate display data associated with the user interface that is provided to the client device involved in generating the input data. The display data can be configured to cause the display device to generate the user interface. In some examples, the display data can be configured to cause the execution of one or more operations by the device that involve updating the user interface at the client device based on user input. This can involve updating the user interface based on user input that specifies one or more parameters to be used to narrow the performance metrics involved in evaluating the models. The client device can then update the user interface based on these one or more parameters.

FIG. 4 is an example of a system architecture 400 for implementing iterative machine learning model optimization through adaptive parameter adjustment, in accordance with one or more embodiments described herein. As shown, the system architecture 400 can be configured to execute one or more operations in an illustrated order. However, it will be understood that the concepts described herein are not limited to that order, and that other orderings of at least some of the operations are contemplated. In some embodiments, the system architecture 400 can be implemented by an analytics server that is the same as, or similar to, the analytics server 102 of FIG. 1. In other embodiments, the system architecture 400 can be implemented by one or more devices that are different from the analytics server 102 or in combination with devices that are different from the analytics server 102.

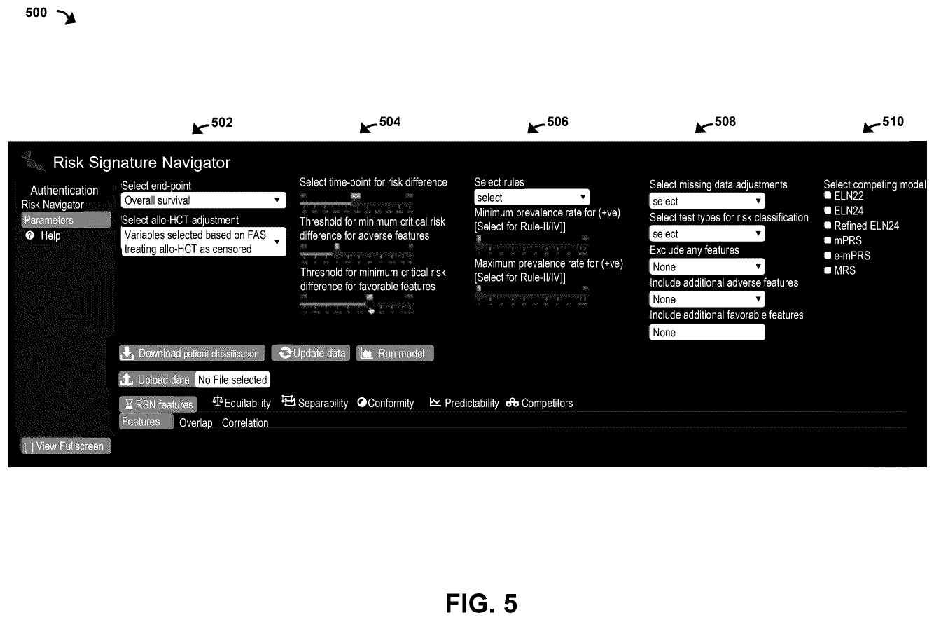

At operation 402, the analytics server can obtain input data associated with at least one user input. For example, the analytics server can obtain input data generated by a client device based on inputs provided by an individual controlling the client device, such as a software developer, data engineer, clinician, or researcher. The input data can indicate an endpoint type associated with a clinical outcome of patients treated with at least one therapeutic agent, such as venetoclax (VEN) and azacitidine (AZA). The endpoint type can include overall survival (OS), event-free survival (EFS), minimal residual disease (MRD), relapse-free survival (RFS), or combinations thereof. Additionally, the input data can indicate one or more configuration parameters including a time-point for evaluating the clinical outcome and a minimum risk-difference threshold. The user controlling the client device can provide these inputs using one or more fields 502 of the user interface 500 (e.g., that is the same as, or similar to, the user interface 500 of FIG. 5). The one or more configuration parameters can further include parameters that indicate a time-point for risk difference, thresholds for minimum critical risk differences between adverse features, and thresholds for minimum critical risk differences for favorable features (using one or more fields 504 of the user interface 500 of FIG. 5), one or more rules, a minimum prevalence rate, a maximum prevalence rate (using one or more fields 506 of the user interface 500 of FIG. 5), one or more techniques that can be implemented to adjust for missing data in one or more datasets as described herein, one or more types of risk classifications, one or more features to include or exclude (using one or more fields 508 of the user interface 500 of FIG. 5), and/or one or more models to be evaluated (using one or more toggle buttons 510 of the user interface 500 of FIG. 5).

At operation 404, the analytics server can determine one or more criteria corresponding to an endpoint based on the endpoint type and the one or more configuration parameters. For example, where the endpoint type is associated with event-free survival, the analytics server can establish criteria such as the absence of disease recurrence, progression, or death at the time-point specified by the configuration parameters. In other examples, where the endpoint type is associated with overall survival, the analytics server can establish criteria for survival rates at the specified time-point. The analytics server can then obtain a dataset including one or more treatment profiles and/or limited treatment profiles that satisfy the one or more criteria. For example, the analytics server can obtain treatment profiles and/or limited treatment profiles that satisfy one or more diagnostic criteria as described herein. In examples, obtaining the treatment profiles and/or limited treatment profiles can involve identifying the relevant profiles in a global patient database or in a refined dataset as described with respect to the global patient database 110 and/or the refined datasets 108 of FIG. 1.

In some embodiments, the analytics server can determine one or more features from among a plurality of candidate features to use when evaluating a plurality of models. For example, the analytics server can analyze biomarkers represented across the treatment profiles to select features (e.g., represented as the results of analyzing flow cytometry (FC), cytogenetic analysis (CYT), fluorescence in situ hybridization (FISH), next-generation sequencing (NGS), and/or AML mutation (e.g., detected through polymerase chain reaction (PCR) tests)). The biomarkers can be selected based on univariate Kaplan-Meier (KM) survival analyses or similar analyses. These biomarkers can be evaluated for their association with clinical outcomes such as overall survival (OS), event-free survival (EFS), minimal residual disease (MRD), and relapse-free survival (RFS). In some embodiments, the analytics server can group the features into a set X, which can each include individual biomarker variables (X_{FC}, X_{CYT}, X_{FISH}, X_{RD}, X_{MUT}, etc.).

In some embodiments, the analytics server can compute, for each candidate feature and for the endpoint type, an endpoint-specific risk difference between patients having the feature and patients lacking the feature at the time-point specified by the configuration parameters. For example, the analytics server can evaluate each candidate biomarker to determine whether the difference in predicted clinical outcome (e.g., overall survival, event-free survival, etc.) between patients exhibiting the biomarker and those not exhibiting the biomarker exceeds a predefined threshold at the specified time-point. The analytics server can then select the one or more features as those candidate features whose endpoint-specific risk difference satisfies the minimum risk-difference threshold. Based on this analysis, the analytics server can select a subset of features (that include, at least in part, biomarker values representing the state of respective patients at one or more points in time) that are clinically relevant and satisfy the user-defined risk-difference criteria.

In some embodiments, the analytics server can address missing data (e.g., missing biomarker data, etc.) in the treatment profiles. For example, the analytics server can implement inverse probability weighting (IPW) based on generalized linear models (GLMs) for complete case analyses. Additionally, missing values can be imputed five times using multiple imputation by chained equations (MICE). The “impute-then-select” approach can allow for imputation to be performed for each biomarker before further analysis. By selecting features as described above, the analytics server can classify biomarkers, or combinations of biomarkers, as either being strongly favorable, moderately favorable, neutral, moderately adverse, and strongly adverse predictors of a given health outcome associated with a corresponding endpoint type and/or configuration.

At operation 406, the analytics server can configure a first model and a second model based on the one or more criteria and the one or more features. For example, the analytics server can configure the first model and the second model by parameterizing the first model and the second model to output, for each treatment profile, a risk stratification into a plurality of risk groups for the endpoint type. The analytics server can set model parameters without recompiling model code, based on the endpoint-specific configuration parameters obtained from the client device. This parameterization can enable the analytics server to adapt the models to varying clinical scenarios and evaluation criteria without requiring recompilation or redevelopment of the underlying model architecture. In some embodiments, the analytics server can select the first model and the second model from among a plurality of models based on a task indicated by the input data, such as classifying treatment profiles in accordance with a risk stratification scheme. The plurality of models can include risk stratification models configured for classification of overall survival rates, event-free survival rates, or combinations thereof. In examples, these models can be based on various architectures, including ELN 22 (European LeukemiaNet 2022), ELN 24 (European LeukemiaNet 2024 (Dohner et al. 2024)), Refined ELN 22 (Refined European LeukemiaNet 2024 (Curtis et al 2024)), mPRS (molecular prognostic risk signatures (Dohner et al 2024)), e-mPRS (extended molecular prognostic risk signature (Islam et al 2024)), and/or MRS (risk signatures developed by Mayo-Clinic (Naseema et al. 2024)). In some examples, these models can include semi-parametric regularized models (e.g., hazard models with ridge penalty, etc.). In examples, hazard models compatible with survival data can additionally, or alternatively, be implemented without loss of generality (e.g., deep survival network, random survival forest, or other semi-parametric models with different penalty terms).

In some embodiments, the analytics server can cause a model development environment to implement one or more stateless training operations to train the first model and the second model to implement risk stratification in accordance with the treatment profiles obtained by the analytics server. In some embodiments, this can include the analytics server causing separate penalized Cox proportional hazards (Cox-PH) regression models to be fit for each biomarker to assess their influence (e.g., their ability to satisfy the endpoint type) on survival outcomes. Tuning parameters for these models can be optimized via cross-validation to prevent overfitting and ensure robust results.

In some embodiments, the analytics server can generate “recycled” predictions, which involve calculating mean marginal probabilities of risk over time using counterfactual arguments (e.g., whether a particular mutation was present or not present). For each treatment profile, hypothetical scenarios can be created where each biomarker is either positive or negative, allowing for comparison of risk profiles under different conditions.

In some embodiments, the analytics server can compute bias-corrected 95% confidence intervals (CIs) for various estimates, marginal risks, and risk differences to quantify uncertainty and improve reliability of the results.

In some embodiments, the analytics server can calculate risk differences (RDs) at a specific point in time (e.g., 6 months, 12 months, 18 months, etc.). In some embodiments, the analytics server can determine the risk differences using a formula:

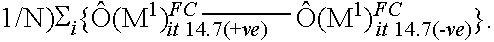

This can allow the analytics server to obtain covariate-level stratification using a 5% risk difference rule to categorize subjects into meaningful subgroups.

In some embodiments, the analytics server can then apply logic-based rules to stratify individual subjects using the above-developed models based on their specific risk profiles derived from the previous steps. This can allow the analytics server to develop personalized risk assessment and clinical decision-making.

At operation 408, the analytics server can execute the first model and the second model on treatment profiles selected from the database based on the endpoint criteria and the subset of features to generate a first set of model outputs and a second set of model outputs. For example, the analytics server can provide treatment profiles as inputs to the first model and the second model, where the treatment profiles can include relevant portions of electronic health records (EHRs), laboratory results, genetic profiles, and other biomarkers relevant to the treatment being evaluated. In some embodiments, the treatment profiles can be preprocessed to remove or update personal health information and/or personally identifiable information before being provided as inputs to the models. The analytics server can obtain the outputs of the models, where the outputs correspond to the classification of each treatment profile into risk categories such as “favorable,” “intermediate,” or “adverse” responses to the at least one therapeutic agent.

At operation 410, the analytics server can compute at least one performance metric value for each of the first model and the second model based on the corresponding set of model outputs. For example, the analytics server can obtain data associated with one or more performance metrics determined based on the performance of the machine learning models once trained and/or evaluated. The performance metric value can include at least one of: a separability metric between the risk groups, a conformity metric between the risk groups and observed clinical outcomes, or a predictability metric at the time-point. At operation 412, the analytics server can then obtain the performance metrics from the model development environment.

At operation 414, the analytics server can generate and provide data associated with a user interface representing the performance metrics to the client device associated with the user that generated the input data. In response to executing the first model and the second model, the analytics server can generate the user interface including an indication of a first performance metric value for the first model and a second performance metric value for the second model, the first performance metric representing the output of the first model and the second performance metric representing the output of the second model. The user interface can include one or more indications of the performance metrics such as overall performance of the one or more models, performance of the one or more models at one or more points in time, etc. In some embodiments, the indication of the first performance metric value can be a first heatmap visually encoding the at least one performance metric value of the first model over a plurality of time-points, the indication of the second performance metric value can be a second heatmap visually encoding the at least one performance metric value of the second model over a plurality of time-points, and the user interface can concurrently present the first heatmap and the second heatmap to enable comparative visual evaluation of the first model and the second model. Optionally, the client device can receive additional input from the user controlling the client device indicating the acceptance or non-acceptance of one or more machine learning models for deployment. For example, the client device can receive input identifying one or more models that achieve satisfactory performance for diagnostic purposes, and as a result can be provided to other client devices controlled by other researchers, clinicians, etc., for use when analyzing treatment profiles.

At operation 416, the analytics server can be configured to deploy one or more machine learning models. For example, the analytics server can be configured to deploy one or more machine learning models for risk stratification that are developed using the model development environment. In some examples, the analytics server can be configured to deploy models configured as described herein that classify treatment profiles corresponding to patients as either favorable, neutral, or not favorable candidates for one or more therapies such as one or more agents configured to target a particular disease (e.g., venetoclax (VEN) and azacitidine (AZA) as configured to target AML). In other examples, the analytics server can configure and deploy models that classify treatment profiles corresponding to any number of patient-level classifications. For example, the analytics server can configure and deploy patient-level classifications that indicate whether a patient is strongly-favorable, favorable, intermediate, adverse, strongly-adverse, or any combination thereof.

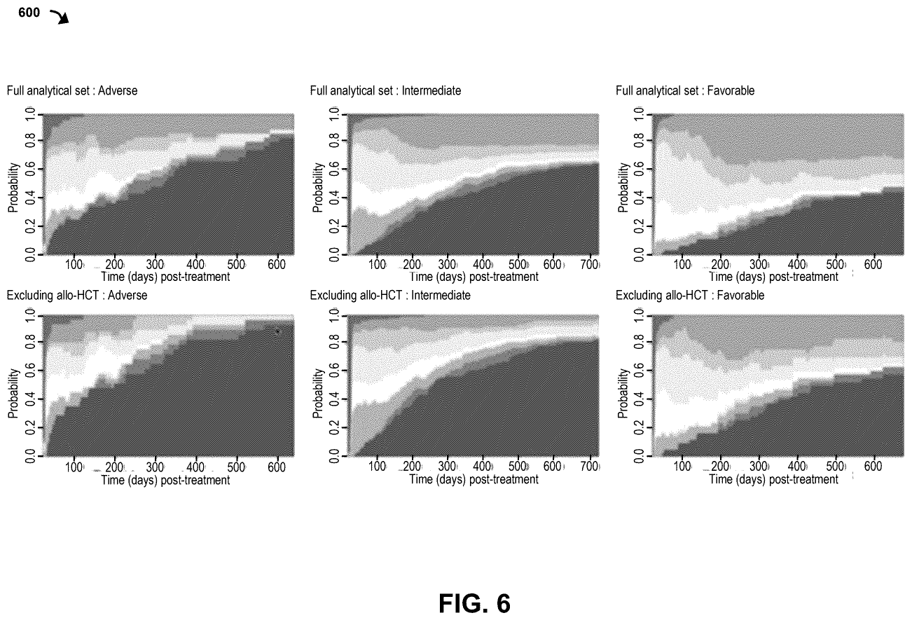

FIG. 6 is an example of a set of heatmaps 600 generated to indicate the evolution of disease states stratified by patient-level classification, in accordance with one or more embodiments described herein. As shown, six heatmaps can be generated to indicate example risk stratifications for treatment profiles that can be configured by the analytics server as described above. In some embodiments, the analytics server can be configured to train models, such as statistics-based models or machine learning-based models as descried herein, to generate the set of heatmaps 600 that indicate patient risk stratifications. For example, an analytics server (e.g., that is the same as, or similar to, the analytics server 102 of FIG. 1) can be configured to obtain datasets including treatment profiles, limited treatment profiles, etc., where each treatment profile includes values corresponding to biomarker levels or medical histories or events, etc., to train statistical models or machine learning algorithms. Once trained, the models can process new patient data to assign risk scores, which are then visualized through the generation of the set of heatmaps 600.

In some embodiments, the heatmaps 600 can include diagrams showing the disease states to illustrate the progression of patients through various stages of disease progression. These stages can indicate the state of a patient at one or more points in time during disease progression. The events can include diagnosis, treatment initiation, follow-up responses (such as CR, CRI, CRH), relapse, and mortality. The heatmaps 600 can visually represent the distribution and transitions of patients having similar treatment profiles across these states, highlighting the differences in outcomes between adverse, intermediate, and favorable groups. For example, the adverse group (e.g., represented by the heatmaps in the first column) can show higher mortality rates, while the favorable group (e.g., represented by the heatmaps in the third column) can show higher rates of complete response (CR). By virtue of generating the heatmaps 600, the analytics server can cause user interfaces to be generated that provide a comprehensive view of how patients with similar treatment profiles may move through different disease states using the models configured by the analytics server. As illustrated, models can be configured to generate outputs that are then used by an analytics server to represent the evolution of disease states stratified by patient-level classification where, for example, patients are receive one or more therapies or not (e.g., receive healthy blood-forming stem cells from a donor to replace their own damaged or diseased stem cells, etc.).

Example embodiments include the following systems and methods.

A system for iteratively optimizing machine learning models through adaptive parameter adjustment includes one or more processors. The processors obtain input data from at least one user that specifies an endpoint type related to a clinical outcome for patients treated with at least one therapeutic agent, along with configuration parameters including a time-point for evaluating the clinical outcome and a minimum risk-difference threshold. The processors determine criteria for an endpoint based on the endpoint type and the configuration parameters. The processors determine which features to use when evaluating multiple models by computing, for each candidate feature and the endpoint type, a risk difference between patients who have the feature and patients who lack the feature at the specified time-point, and selecting features whose risk difference meets the minimum threshold. Each feature corresponds to diagnostic criteria in a database of treatment profiles. The processors configure a first model and a second model by parameterizing them to output risk stratifications into multiple risk groups for the endpoint type and setting model parameters without recompiling model code, based on the endpoint-specific configuration parameters obtained from a client device. The processors execute the first and second models on treatment profiles selected from the database based on the endpoint criteria and the selected features to generate outputs. For each model, the processors compute at least one performance metric value based on the corresponding outputs. In response to executing the models, the processors generate a user interface that displays a first performance metric value for the first model and a second performance metric value for the second model, where the first performance metric represents the output of the first model and the second performance metric represents the output of the second model. The processors provide the user interface to a device that generated the input data to show the first and second performance metric values.

In the system above, the performance metric value can include at least one of: a separability metric between the risk groups, a conformity metric between the risk groups and observed clinical outcomes, or a predictability metric at the specified time-point.

In the system above, the first performance metric value can be displayed as a first heatmap that visually encodes the performance metric value of the first model over multiple time-points, the second performance metric value can be displayed as a second heatmap that visually encodes the performance metric value of the second model over multiple time-points, and the user interface can present both heatmaps simultaneously to enable comparative visual evaluation of the first model and the second model.

In the system above, the processors can determine the criteria for the endpoint by determining a period of time associated with the endpoint type, and the processors can obtain the first model and the second model from a device that updates the models after executing operations to update the first and second models to cause them to generate outputs in accordance with the period of time.

In the system above, the processors can update the first model and the second model using a training database by obtaining treatment profiles representing states of patients being treated, executing operations to update aspects of each treatment profile to de-identify at least a portion of each treatment profile, and generating the training database after de-identifying at least a portion of each treatment profile.

In the system above, the processors can determine the features by determining multiple features, where each feature corresponds to an element of diagnostic criteria, and where the diagnostic criteria correspond to at least one of chromosomal analysis, flow cytometry, fluorescence in situ hybridization, or next generation sequencing.

In the system above, the processors can determine the multiple features by determining features to be excluded that are represented by the treatment profiles, and determining the multiple features based on the input data and the excluded features.

In the system above, the processors can configure the first and second models by obtaining them from among multiple models, where the multiple models include a first risk stratification model configured to output overall survival rates or a second risk stratification model configured to output event-free survival rates.

In the system above, the processors can obtain patient data associated with a supplemental patient database from a device that generated the input data, and update the treatment profiles based on the supplemental patient database to include supplemental treatment profiles from the supplemental patient database.

A method for iteratively optimizing machine learning models through adaptive parameter adjustment is performed by one or more processors. The method includes obtaining input data from at least one user that specifies an endpoint type related to a clinical outcome for patients treated with at least one therapeutic agent, along with configuration parameters including a time-point for evaluating the clinical outcome and a minimum risk-difference threshold. The method includes determining criteria for an endpoint based on the endpoint type and the configuration parameters. The method includes determining which features to use when evaluating multiple models by computing, for each candidate feature and the endpoint type, a risk difference between patients who have the feature and patients who lack the feature at the specified time-point, and selecting features whose risk difference meets the minimum threshold. Each feature corresponds to diagnostic criteria in a database of treatment profiles. The method includes configuring a first model and a second model by parameterizing them to output risk stratifications into multiple risk groups for the endpoint type and setting model parameters without recompiling model code, based on the endpoint-specific configuration parameters obtained from a client device. The method includes executing the first and second models on treatment profiles selected from the database based on the endpoint criteria and the selected features to generate outputs. For each model, the method includes computing at least one performance metric value based on the corresponding outputs. In response to executing the models, the method includes generating a user interface that displays a first performance metric value for the first model and a second performance metric value for the second model, where the first performance metric represents the output of the first model and the second performance metric represents the output of the second model. The method includes providing the user interface to a device that generated the input data to show the first and second performance metric values.

In the method above, the performance metric value can include at least one of: a separability metric between the risk groups, a conformity metric between the risk groups and observed clinical outcomes, or a predictability metric at the specified time-point.

In the method above, the first performance metric value can be displayed as a first heatmap that visually encodes the performance metric value of the first model over multiple time-points, the second performance metric value can be displayed as a second heatmap that visually encodes the performance metric value of the second model over multiple time-points, and the user interface can present both heatmaps simultaneously to enable comparative visual evaluation of the first model and the second model.

In the method above, determining the criteria for the endpoint can include determining a period of time associated with the endpoint type, and the method can further include obtaining the first model and the second model from a device that updates the models after executing operations to update the first and second models to cause them to generate outputs in accordance with the period of time.

In the method above, updating the first model and the second model using a training database can include obtaining treatment profiles representing states of patients being treated, executing operations to update aspects of each treatment profile to de-identify at least a portion of each treatment profile, and generating the training database after de-identifying at least a portion of each treatment profile.

In the method above, determining the features can include determining multiple features, where each feature corresponds to an element of diagnostic criteria, and where the diagnostic criteria correspond to at least one of chromosomal analysis, flow cytometry, fluorescence in situ hybridization, or next generation sequencing.

In the method above, determining the multiple features can include determining features to be excluded that are represented by the treatment profiles, and determining the multiple features based on the input data and the excluded features.

In the method above, executing the first and second models can include obtaining them from among multiple models, where the multiple models include a first risk stratification model configured to output overall survival rates or a second risk stratification model configured to output event-free survival rates.

In the method above, the method can further include obtaining patient data associated with a supplemental patient database from a device that generated the input data, and updating the treatment profiles based on the supplemental patient database to include supplemental treatment profiles from the supplemental patient database.

One or more non-transitory computer-readable mediums store instructions that, when executed by one or more processors, cause the processors to obtain input data from at least one user that specifies an endpoint type related to a clinical outcome for patients treated with at least one therapeutic agent, along with configuration parameters including a time-point for evaluating the clinical outcome and a minimum risk-difference threshold. The instructions cause the processors to determine criteria for an endpoint based on the endpoint type and the configuration parameters. The instructions cause the processors to determine which features to use when evaluating multiple models by computing, for each candidate feature and the endpoint type, a risk difference between patients who have the feature and patients who lack the feature at the specified time-point, and selecting features whose risk difference meets the minimum threshold. Each feature corresponds to diagnostic criteria in a database of treatment profiles. The instructions cause the processors to configure a first model and a second model by parameterizing them to output risk stratifications into multiple risk groups for the endpoint type and setting model parameters without recompiling model code, based on the endpoint-specific configuration parameters obtained from a client device. The instructions cause the processors to execute the first and second models on treatment profiles selected from the database based on the endpoint criteria and the selected features to generate outputs. For each model, the instructions cause the processors to compute at least one performance metric value based on the corresponding outputs. In response to executing the models, the instructions cause the processors to generate a user interface that displays a first performance metric value for the first model and a second performance metric value for the second model, where the first performance metric represents the output of the first model and the second performance metric represents the output of the second model. The instructions cause the processors to provide the user interface to a device that generated the input data to show the first and second performance metric values.

In the computer-readable mediums above, the performance metric value can include at least one of: a separability metric between the risk groups, a conformity metric between the risk groups and observed clinical outcomes, or a predictability metric at the specified time-point.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Some embodiments of the present disclosure are described herein in connection with a threshold. As described herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system for iterative machine learning model optimization through adaptive parameter adjustment, the system comprising:
one or more processors configured to:
obtain input data associated with at least one user input indicating (i) an endpoint type associated with a clinical outcome of patients treated with at least one therapeutic agent and (ii) one or more configuration parameters including a time-point for evaluating the clinical outcome and a minimum risk-difference threshold;
determine one or more criteria corresponding to an endpoint based on the endpoint type and the one more configuration parameters;
determine one or more features from among a plurality of candidate features to use when evaluating a plurality of models, each feature corresponding to diagnostic criteria included in a database representing a plurality of treatment profiles, the determining comprising:
computing, for each candidate feature and for the endpoint type, an endpoint-specific risk difference between patients having the feature and patients lacking the feature at the time-point; and
selecting the one or more features as those candidate features whose endpoint-specific risk difference satisfies the minimum risk-difference threshold;
configure a first model and a second model based on the one or more criteria and the one or more features, the configuring comprising:
parameterizing the first model and the second model to output, for each treatment profile, a risk stratification into a plurality of risk groups for the endpoint type; and
setting model parameters without recompiling model code, based on the endpoint-specific configuration parameters obtained from the client device;
execute the first model and the second model on treatment profiles selected from the database on the endpoint criteria and the subset of features to generate a first set of model outputs and a second set of model outputs;
for each of the first model and the second model, compute at least one performance metric value based on the corresponding set of model outputs;
in response to executing the first model and the second model, generate, based on the at least one performance metric value, a user interface comprising an indication of a first performance metric value for the first model and a second performance metric for the second model, the first performance metric representing the output of the first model and the second performance metric representing the output of the second model; and
provide the user interface to a device involved in generating the input data to indicate the first performance metric value and the second performance metric value.

2. The system of claim 1, wherein the performance metric value including at least one of: a separability metric between the risk groups, a conformity metric between the risk groups and observed clinical outcomes, or a predictability metric at the time-point.

3. The system of claim 1, wherein the indication of the first performance metric value is a first heatmap visually encoding the at least one performance metric value of the first model over a plurality of time-points, the indication of the second performance metric value is a second heatmap visually encoding the at least one performance metric value of the second model over a plurality of time-points, and the user interface concurrently presents the first heatmap and the second heatmap to enable comparative visual evaluation of the first model and the second model.

4. The system of claim 1, wherein the one or more processors configured to determine the one or more criteria corresponding to the endpoint are configured to:
determine a period of time associated with the endpoint type,
wherein the one or more processors are further configured to:
in response to execution of one or more operations to update the first model and the second model to cause the first model and the second model to generate outputs in accordance with the period of time, obtain the first model and the second model from a device involved in updating the first model and the second model.

5. The system of claim 3, wherein the one or more processors configured to update the first model and the second model using the training database are configured to:
obtain a plurality of treatment profiles representing states of patients being treated;
execute one or more operations to update one or more aspects each treatment profile of the plurality of treatment profiles to de-identify at least a portion of each treatment profile; and
generate the training database in response to de-identifying the at least a portion of each treatment profile.

6. The system of claim 1, wherein the one or more processors configured to determine the one or more features are configured to:
determine a plurality of features, each feature of the plurality of features corresponding to an element of the diagnostic criteria, where the diagnostic criteria corresponds to at least one of chromosomal analysis, flow cytometry, fluorescence in situ hybridization, or next generation sequencing.

7. The system of claim 5, wherein the one or more processors configured to determine the plurality of features are configured to:
determine one or more features to be excluded that are represented by the plurality of treatment profiles; and
determine the plurality of features based on the input and the excluded features.

8. The system of claim 1, wherein the one or more processors configured to configure the first model, and the second model are configured to:
obtain the first model and the second model from among a plurality of models, the plurality of models comprising a first risk stratification model configured to output overall survival rates, or a second risk stratification model configured to output event-free survival rates.

9. The system of claim 1, wherein the one or more processors are further configured to:
obtain patient data associated with a supplemental patient database from a device involved in generating the input data associated with at least one user input; and
update the plurality of treatment profiles based on the supplemental patient database to supplemental treatment profiles included in the supplemental patient database.

10. A method for iterative machine learning model optimization through adaptive parameter adjustment, the method comprising:
obtaining, by one or more processors, input data associated with at least one user input indicating (i) an endpoint type associated with a clinical outcome of patients treated with at least one therapeutic agent and (ii) one or more configuration parameters including a time-point for evaluating the clinical outcome and a minimum risk-difference threshold;
determining, by the one or more processors, one or more criteria corresponding to an endpoint based on the endpoint type and the one or more configuration parameters;
determining, by the one or more processors, one or more features from among a plurality of candidate features to use when evaluating a plurality of models, each feature corresponding to diagnostic criteria included in a database representing a plurality of treatment profiles, the determining comprising:
computing, by the one or more processors, for each candidate feature and for the endpoint type, an endpoint-specific risk difference between patients having the feature and patients lacking the feature at the time-point; and
selecting, by the one or more processors, the one or more features as those candidate features whose endpoint-specific risk difference satisfies the minimum risk-difference threshold;
configuring, by the one or more processors, a first model and a second model based on the one or more criteria and the one or more features, the configuring comprising:
parameterizing, by the one or more processors, the first model and the second model to output, for each treatment profile, a risk stratification into a plurality of risk groups for the endpoint type; and
setting, by the one or more processors, model parameters without recompiling model code, based on the endpoint-specific configuration parameters obtained from a client device;
executing, by the one or more processors, the first model and the second model on treatment profiles selected from the database based on the endpoint criteria and the subset of features to generate a first set of model outputs and a second set of model outputs;
for each of the first model and the second model, computing, by the one or more processors, at least one performance metric value based on the corresponding set of model outputs;
in response to executing the first model and the second model, generating, by the one or more processors based on the at least one performance metric value, a user interface comprising an indication of a first performance metric value for the first model and a second performance metric value for the second model, the first performance metric representing the output of the first model and the second performance metric representing the output of the second model; and
providing, by the one or more processors, the user interface to a device involved in generating the input data to indicate the first performance metric value and the second performance metric value.

11. The method of claim 10, wherein the performance metric value includes at least one of: a separability metric between the risk groups, a conformity metric between the risk groups and observed clinical outcomes, or a predictability metric at the time-point.

12. The method of claim 10, wherein the indication of the first performance metric value is a first heatmap visually encoding the at least one performance metric value of the first model over a plurality of time-points, the indication of the second performance metric value is a second heatmap visually encoding the at least one performance metric value of the second model over a plurality of time-points, and the user interface concurrently presents the first heatmap and the second heatmap to enable comparative visual evaluation of the first model and the second model.

13. The method of claim 10, wherein determining, by the one or more processors, the one or more criteria corresponding to the endpoint comprises:
determining a period of time associated with the endpoint type,
wherein the method further comprises:
in response to execution of one or more operations to update the first model and the second model to cause the first model and the second model to generate outputs in accordance with the period of time, obtaining, by the one or more processors, the first model and the second model from a device involved in updating the first model and the second model.

14. The method of claim 13, wherein updating the first model and the second model using a training database comprises:
obtaining, by the one or more processors, a plurality of treatment profiles representing states of patients being treated;
executing, by the one or more processors, one or more operations to update one or more aspects of each treatment profile of the plurality of treatment profiles to de-identify at least a portion of each treatment profile; and
generating, by the one or more processors, the training database in response to de-identifying the at least a portion of each treatment profile.

15. The method of claim 10, wherein determining, by the one or more processors, the one or more features comprises:
determining a plurality of features, each feature of the plurality of features corresponding to an element of the diagnostic criteria, where the diagnostic criteria corresponds to at least one of chromosomal analysis, flow cytometry, fluorescence in situ hybridization, or next generation sequencing.

16. The method of claim 15, wherein determining, by the one or more processors, the plurality of features comprises:
determining one or more features to be excluded that are represented by the plurality of treatment profiles; and
determining the plurality of features based on the input data and the excluded features.

17. The method of claim 10, wherein executing, by the one or more processors, the first model and the second model comprises:
obtaining the first model and the second model from among a plurality of models, the plurality of models comprising a first risk stratification model configured to output overall survival rates, or a second risk stratification model configured to output event-free survival rates.

18. The method of claim 10, further comprising:
obtaining, by the one or more processors, patient data associated with a supplemental patient database from a device involved in generating the input data associated with at least one user input; and
updating, by the one or more processors, the plurality of treatment profiles based on the supplemental patient database to include supplemental treatment profiles included in the supplemental patient database.

19. One or more non-transitory computer-readable mediums storing instructions thereon that, when executed by one or more processors, cause the one or more processors to:
obtain input data associated with at least one user input indicating (i) an endpoint type associated with a clinical outcome of patients treated with at least one therapeutic agent and (ii) one or more configuration parameters including a time-point for evaluating the clinical outcome and a minimum risk-difference threshold;
determine one or more criteria corresponding to an endpoint based on the endpoint type and the one or more configuration parameters;
determine one or more features from among a plurality of candidate features to use when evaluating a plurality of models, each feature corresponding to diagnostic criteria included in a database representing a plurality of treatment profiles, the determining comprising:
computing, for each candidate feature and for the endpoint type, an endpoint-specific risk difference between patients having the feature and patients lacking the feature at the time-point; and
selecting the one or more features as those candidate features whose endpoint-specific risk difference satisfies the minimum risk-difference threshold;
configure a first model and a second model based on the one or more criteria and the one or more features, the configuring comprising:
parameterizing the first model and the second model to output, for each treatment profile, a risk stratification into a plurality of risk groups for the endpoint type; and
setting model parameters without recompiling model code, based on the endpoint-specific configuration parameters obtained from a client device;
execute the first model and the second model on treatment profiles selected from the database based on the endpoint criteria and the subset of features to generate a first set of model outputs and a second set of model outputs;
for each of the first model and the second model, compute at least one performance metric value based on the corresponding set of model outputs;
in response to executing the first model and the second model, generate, based on the at least one performance metric value, a user interface comprising an indication of a first performance metric value for the first model and a second performance metric value for the second model, the first performance metric representing the output of the first model and the second performance metric representing the output of the second model; and
provide the user interface to a device involved in generating the input data to indicate the first performance metric value and the second performance metric value.

20. The one or more non-transitory computer-readable mediums of claim 19, wherein the performance metric value includes at least one of: a separability metric between the risk groups, a conformity metric between the risk groups and observed clinical outcomes, or a predictability metric at the time-point.
